(19) United States
(12) Patent Application Publication
(10) Pub. No.: US 2025/0022134 A1
(43) Pub. Date: Jan. 16, 2025

(54) APPARATUS AND METHOD FOR AUDITING OF ARTIFICIAL INTELLIGENCE-BASED MEDICAL IMAGE ENHANCEMENT

(71) Applicant: CLARIPI INC., Seoul (KR)

(72) Inventors: Chul Kyun AHN, Seoul (KR); Jong Hyo KIM, Seoul (KR)

(21) Appl. No.: 18/771,496

(22) Filed: Jul. 12, 2024

(30) Foreign Application Priority Data
Jul. 13, 2023 (KR) 10-2023-0091369

(51) Int. Cl. G06T 7/00

(52) U.S. Cl. CPC G06T 7/0014; G06T 2207/20021; G06T 2207/20072; G06T 2207/20081; G06T 2207/30004; G06T 2207/30168

(57) ABSTRACT

Disclosed is a method of auditing of artificial intelligence-based medical image enhancement, including: performing preprocessing to generate a comparison image based on an input image and an output image received from a medical image enhancement device; generating a heatmap image to generate a hallucination heatmap image including a hallucination region in the comparison image by inputting the comparison image to a deep learning model trained in advance; calculating an error risk to calculate a hallucination risk for the hallucination region based on pixel values of the hallucination heatmap image; and providing auditing information to provide the auditing information for auditing accuracy of the output image based on the calculated hallucination risk to a user.

### CROSS REFERENCE TO RELATED APPLICATION

Priority to Korean patent application number 10-2023-0091369 filed on Jul. 13, 2023 the entire disclosure of which is incorporated by reference herein, is claimed.

### BACKGROUND OF THE INVENTION

### Field of the Invention

The disclosure relates to an apparatus and method for auditing of an artificial intelligence-based medical image enhancement device, and more particularly to an apparatus and method for auditing of artificial intelligence-based medical image enhancement device, which compares an input image input to a medical image enhancement device and a result output from the medical image enhancement device.

### Description of the Related Art

In general, an X-ray, a computed tomography (CT), a magnetic resonance imaging (MRI), and the like medical apparatuses are used to acquire medical images. In modern medicine, the medical images acquired through such medical apparatuses are used as a very important basis for the presence and characteristics of lesions to make decisions in a process of diagnosing and treating a patient.

A related art to medical image processing has been disclosed in Korean Patent Publication No. 2014-0134903 (titled “METHOD AND APPARATUS FOR IMPROVING QUALITY OF MEDICAL IMAGE,” and registered on Nov. 25, 2014). The related art is to provide a high-quality medical image to a reading doctor by removing noise from a medical image.

With recent advancement of artificial intelligence (AI) technology, various technologies have been researched and developed in medical industry to convert low-quality medical images into high-quality medial images. However, artificial intelligence-based medical image enhancement has a problem that the AI may generate a hallucination not based on given data. In particular, medical images in a medical field are used as a very important basis for the presence and characteristics of lesions to make decisions in a process of diagnosing and treating a patient. Accordingly, a technology for auditing whether the hallucination is generated is required.

### SUMMARY OF THE INVENTION

An aspect of the disclosure is to provide an apparatus and method for auditing artificial intelligence-based medical image enhancement device, which audits whether a hallucination is generated in a medical image enhancement process.

According to an embodiment of the disclosure, a method of auditing of artificial intelligence-based medical image enhancement includes: performing preprocessing to generate a comparison image based on an input image and an output image received from a medical image enhancement device; generating a heatmap image to generate a hallucination heatmap image including a hallucination region in the comparison image by inputting the comparison image to a deep learning model trained in advance; calculating an error risk to calculate a hallucination risk for the hallucination region based on pixel values of the hallucination heatmap image; and providing auditing information to provide the auditing information for auditing accuracy of the output image based on the calculated hallucination risk to a user.

The performance of the preprocessing may include: receiving the input image and the output image; generating a difference image between the input image and the output image; applying a convolution operation with a preset kernel to the difference image; and generating the comparison image by applying a predefined threshold to a result of the convolution operation.

The performance of the preprocessing may include: receiving the input image and the output image; calculating a local structural similarity index measure (SSIM) for each pixel of the input image and the output image; and generating the comparison image by generating an image based on the local SSIM and applying a predefined threshold to the local SSIM.

The generation of the heatmap image may include: receiving the comparison image; and applying the comparison image to the deep learning model, which divide the comparison image into a normal image and an abnormal image, to output a hallucination region in a heatmap format.

In training the deep learning model, the method may further include: generating a plurality of output images by inputting the plurality of input images to the medical image enhancement device, labeling the plurality of output images divisionally with normal images and abnormal images to make up a training data set, and inputting the training data set to the deep learning model so that the deep learning model can be trained to output a hallucination heatmap image for the hallucination region.

In the training, the deep learning model is trained to compare the normal image and the abnormal image, and identify a hallucination region based on a changed region in the abnormal image compared to the normal image.

The deep learning model may be provided as a single model to identify the hallucination region from the comparison image upon the comparison image being provided, and apply a heatmap format to the hallucination region.

The deep learning model may include: a first deep learning model configured to identify the hallucination region from the comparison image upon the comparison image being provided; and a second deep learning model linked to the first deep learning model and configured to apply a heatmap format to the hallucination region.

The calculation of the error risk may include calculating the hallucination risk based on at least one of a maximum pixel value or a pixel value sum of the hallucination heatmap image.

The provision of the auditing information may include: sorting the plurality of hallucination heatmap images, in which the hallucination risks have been calculated, in order of high hallucination risk, and providing the input image, the output image, and the hallucination heatmap image, in which the hallucination risk is high, to the user.

Meanwhile, according to an embodiment of the disclosure, an apparatus for auditing of artificial intelligence-based medical image enhancement includes: a preprocessing module configured to generate a comparison image based on an input image and an output image received from a medical image enhancement device; a heatmap generation module configured to generate a hallucination heatmap image including a hallucination region in the comparison image by inputting the comparison image to a deep learning model trained in advance; and a risk calculation module configured to calculate a hallucination risk for the hallucination region based on pixel values of the hallucination heatmap image, wherein auditing information for auditing accuracy of the output image based on the calculated hallucination risk is provided to a user.

The preprocessing module may be configured to: receive the input image and the output image; generate a difference image between the input image and the output image; apply a convolution operation with a preset kernel to the difference image; and generate the comparison image by applying a predefined threshold to a result of the convolution operation.

The preprocessing module may be configured to: receive the input image and the output image; calculate a local structural similarity index measure (SSIM) for each pixel of the input image and the output image; and generate the comparison image by generating an image based on the local SSIM and applying a predefined threshold to the local SSIM.

The heatmap generation module may be configured to: receive the comparison image; and apply the comparison image to the deep learning model, which divide the comparison image into a normal image and an abnormal image, to output a hallucination region in a heatmap format.

In training the deep learning model, a plurality of output images may be generated by inputting the plurality of input images to the medical image enhancement device, the plurality of output images may be divisionally labeled with normal images and abnormal images to make up a training data set, and the training data set may be input to the deep learning model so that the deep learning model can be trained to output a hallucination heatmap image for the hallucination region

The deep learning model may be trained to compare the normal image and the abnormal image, and identify a hallucination region based on a changed region in the abnormal image compared to the normal image.

The deep learning model may be provided as a single model to identify the hallucination region from the comparison image upon the comparison image being provided, and apply a heatmap format to the hallucination region.

The deep learning model may include: a first deep learning model configured to identify the hallucination region from the comparison image upon the comparison image being provided; and a second deep learning model linked to the first deep learning model and configured to apply a heatmap format to the hallucination region.

The risk calculation module may be configured to calculate the hallucination risk based on at least one of a maximum pixel value or a pixel value sum of the hallucination heatmap image.

In providing the auditing information, the plurality of hallucination heatmap images, in which the hallucination risks have been calculated, may be sorted in order of high hallucination risk, and the input image, the output image, and the hallucination heatmap image, in which the hallucination risk is high, may be provided to the user.

According to the disclosure, the apparatus and method for the auditing of the artificial intelligence-based medical image enhancement makes it possible to identify whether the hallucination is involved in the results output from the medical image enhancement device, thereby ensuring the safe operations of the medical image enhancement device.

The technical effects of the disclosure are not limited to the aforementioned effects, and other unmentioned technical effects may become apparent to those skilled in the art from the following description.

### BRIEF DESCRIPTION OF THE DRAWINGS

FIG. 1 is a conceptual view schematically showing an image enhancement auditing apparatus according to an embodiment,

FIG. 2 is a flowchart showing an auditing method using an image enhancement auditing apparatus according to an embodiment,

FIG. 3 is a conceptual view showing a preprocessing module of an image enhancement auditing apparatus according to an embodiment,

FIG. 4 is a conceptual view showing a first preprocessing method of an image enhancement auditing apparatus according to an embodiment,

FIG. 5 is a conceptual showing a second preprocessing method of an image enhancement auditing apparatus according to an embodiment,

FIG. 6 is a conceptual view showing a method of generating a heatmap image in an auditing method using an image enhancement auditing apparatus according to an embodiment,

FIG. 7 is a conceptual view showing a method of training a deep learning model that generates a heatmap image in an auditing method using an image enhancement auditing apparatus according to an embodiment,

FIG. 8 is a conceptual view showing a method of calculating an enhancement risk based on a heatmap image in an auditing method using an image enhancement auditing apparatus according to an embodiment, and

FIG. 9 is a flowchart showing a method of providing auditing information in an image enhancement auditing apparatus according to an embodiment.

### DETAILED DESCRIPTION OF THE INVENTION

Hereinafter, embodiments of the disclosure will be described in detail with reference to the accompanying drawings. However, the disclosure is not limited to the embodiments set forth herein, but may be implemented in various different ways. The embodiments are provided to only complete the disclosure and allow those skilled in the art to understand the category of the disclosure. In the accompanying drawings, the shape, etc. of an element may be exaggerated for clear description, and like numerals refer to like elements.

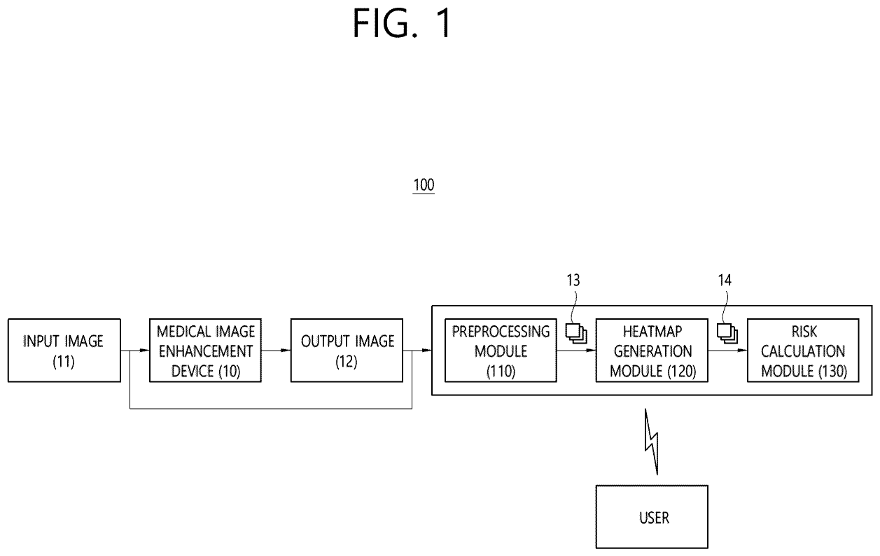

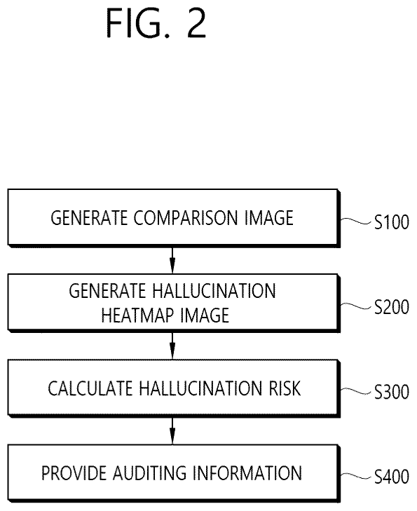

FIG. 1 is a conceptual view schematically showing an image enhancement auditing apparatus according to an embodiment, and FIG. 2 is a flowchart showing an auditing method using an image enhancement auditing apparatus according to an embodiment.

As shown in FIGS. 1 and 2, an image enhancement auditing apparatus 100 according to an embodiment outputs auditing information for auditing whether a hallucination is generated in an image enhancement process for a medical image, while interworking with a medical image enhancement device 10.

In general, the medical image enhancement device 10 convert low-quality medical images into high-quality medial images based on artificial intelligence (AI). However, in the image enhancement process, there is a problem that the AI may generate a hallucination not based on given data.

Thus, the image enhancement auditing apparatus 100 allows a user to audit an image enhancement process, based on an input image 11 input to the medical image enhancement device 10, and an output image 12 output from the medical image enhancement device 10.

Both the input image 11 input to the medical image enhancement device 10 and the output image 12 output from the medical image enhancement device 10 may be provided to the image enhancement auditing apparatus 100. Thus, the image enhancement auditing apparatus 100 may include a preprocessing module 110 that performs preprocessing to generate a comparison image 13 based on comparison between the input image 11 and the output image 12 (S100).

Then, the image enhancement auditing apparatus 100 generates a hallucination heatmap image 14 (hereinafter referred to as a heatmap image) based on the input image 11 and the comparison image 13 (S200). The image enhancement auditing apparatus 100 may include a heatmap generation module 120, and the heatmap generation module 120 may include a deep learning model trained in advance. Thus, the heatmap generation module 120 generates the heatmap image 14 by inputting the comparison image 13 to the deep learning model trained in advance.

In addition, the image enhancement auditing apparatus 100 calculates a hallucination risk based on the heatmap image 14 provided from the heatmap generation module 120 (S300). In this case, the image enhancement auditing apparatus 100 may include a risk calculation module 130, and the risk calculation module 130 may calculate the hallucination risk based on a pixel value of the heatmap image.

Finally, the image enhancement auditing apparatus 100 may provide an auditing result to a user based on the hallucination risk provided from the risk calculation module 130 (S400).

Meanwhile, an auditing method using the image enhancement auditing apparatus will be described in detail with reference to the accompanying drawings. However, the foregoing elements will not be repetitively described, and assigned with the same reference numerals.

### <How to Perform Preprocessing>

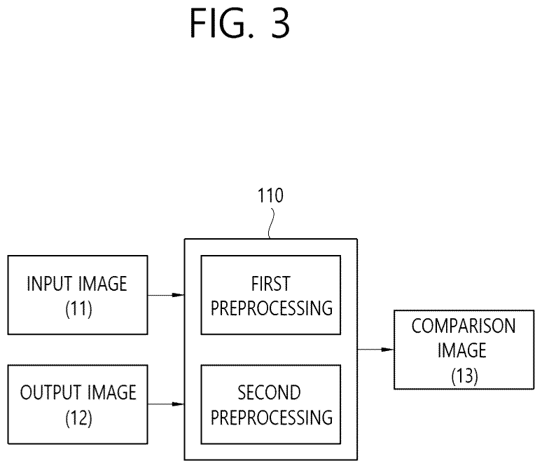

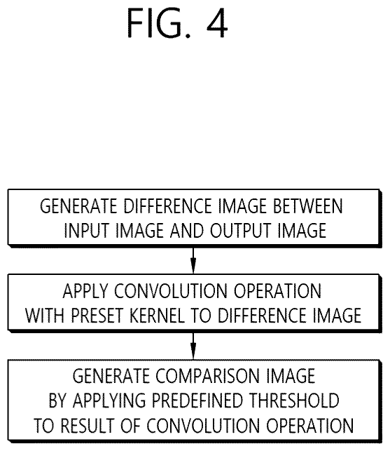

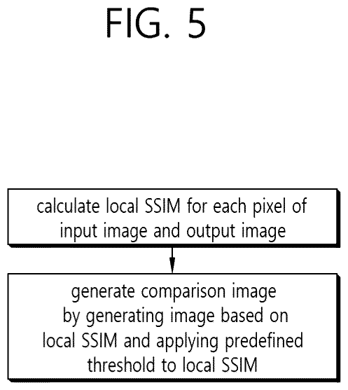

FIG. 3 is a conceptual view showing a preprocessing module of an image enhancement auditing apparatus according to an embodiment, and FIG. 4 is a conceptual view showing a first preprocessing method of an image enhancement auditing apparatus according to an embodiment. In addition, FIG. 5 is a conceptual view showing a second preprocessing method of an image enhancement auditing apparatus according to an embodiment.

As shown in FIGS. 3 to 5, the image enhancement auditing apparatus 100 according to an embodiment includes the preprocessing module 110.

Both the input image 11 and the output image 12 may be provided to the preprocessing module 110. Thus, the preprocessing module 110 may generate the comparison image 13 based on the input image 11 and the output image 12. To generate the comparison image 13, a first preprocessing method and a second preprocessing method may be used.

For example, according to the first preprocessing method, when the input image 11 and the output image 12 are provided, the comparison image 13 may be generated based on a difference image between the input image 11 and the output image 12. In this case, the preprocessing module 110 may obtain a difference image between the input image 11 and the output image 12, and apply a convolution operation with a predefined kernel to the difference image. Then, a predefined threshold is applied to a result of the convolution operation, thereby generating the comparison image 13.

For example, according to the second preprocessing method, when the input image 11 and the output image 12 are provided, the comparison image 13 may be generated based on a similarity index. In this case, the preprocessing module 110 calculates a local structural similarity index measure (SSIM) for each pixel of the input image 11 and the output image 12. Then, the preprocessing module 110 may generate an image based on the local SSIM, and apply a predefined threshold to the local SSIM, thereby generating the comparison image 13.

Then, the preprocessing module 110 may provide the input image 11 and the comparison image 13 to the heatmap generation module 120.

### <How to Generate Hallucination Heatmap>

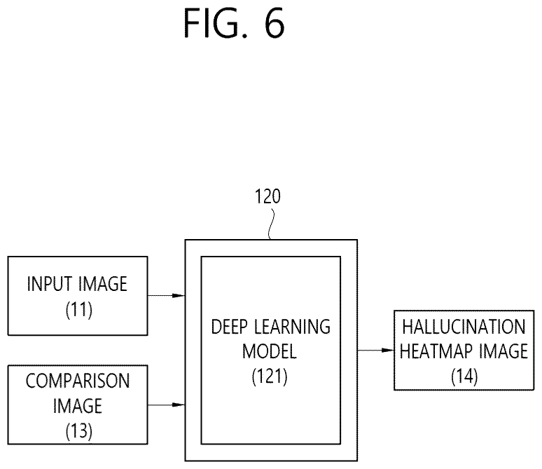

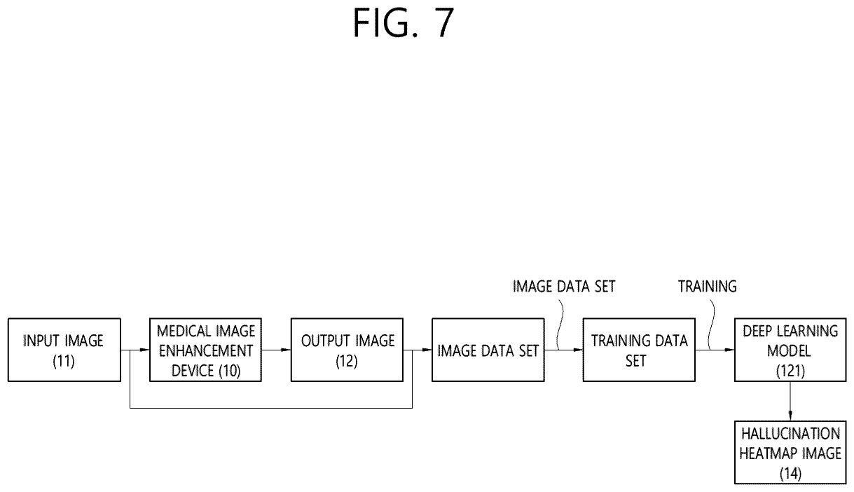

FIG. 6 is a conceptual view showing a method of generating a heatmap image in an auditing method using an image enhancement auditing apparatus according to an embodiment, and FIG. 7 is a conceptual view showing a method of training a deep learning model that generates a heatmap image in an auditing method using an image enhancement auditing apparatus according to an embodiment.

As shown in FIGS. 6 and 7, the image enhancement auditing apparatus 100 according to an embodiment includes the heatmap generation module 120.

The heatmap generation module 120 inputs the input image 11 and the comparison image 13 to a deep learning model 121 trained in advance, so that the deep learning model 121 can generate the heatmap image 14 in which a hallucination is expressed in a heatmap format. In other words, the heatmap generation module 120 may output a hallucination region as visual graphics in the form of heat distribution. The deep learning model 121 may be trained in advance to generate a heatmap image selectively based on the hallucination region.

The deep learning model 121 is trained as follows. First, a large amount of medical image data sets is collected. Here, the medical image data sets may include, but not limited to, medical image data sets acquired from various medical apparatuses such as X-ray, CT and MRI apparatuses.

Then, in training the deep learning model 121, the medical image data sets are input as a plurality of input images 11 to the artificial intelligence-based medical image enhancement device 10, thereby generating a plurality of output images 12. Thus, an image data set for the input image 11 and the output image 12 is provided.

Then, in training the deep learning model 121, labeling is performed for the image data set. In the labeling, it is distinguished whether the output image 12 is normally converted in the image enhancement process, and whether the hallucination is involved due to a hallucinatory process. Here, an image obtained by normally enhancing the quality of the output mage 12 may be a normal image, and an image containing a hallucination may be an abnormal image. Thus, the normal image and the abnormal image make up a training data set.

Further, in training the deep learning model 121, the training data set is used to train the deep learning model 121. In this case, the normal image and the abnormal image are compared in training the deep learning model 121, and a changed region in the abnormal image compared to the normal image is identified as the hallucination region. Then, the deep learning model 121 is trained to output a heatmap image 14 for the hallucination region.

The deep learning model 121 may be provided as a single model to identify a hallucination region and generate a heatmap for the identified hallucination region. Alternatively, the deep learning model 121 may operate to link a plurality of models together so that a first deep learning model can identify the hallucination region and a second deep learning model can generate a heatmap image for the identified hallucination region.

Thus, when the input image 11 and the comparison image 13 are provided by the preprocessing module 110, the heatmap generation module 120 may input the input image 11 and the comparison image 13 to the deep learning model 121 trained in advance, and generate the heatmap image 14. Then, the heatmap generation module 120 may provide the generated heatmap image 14 to the risk calculation module 130.

### <How to Calculate Hallucination Risk and Provide Auditing Information>

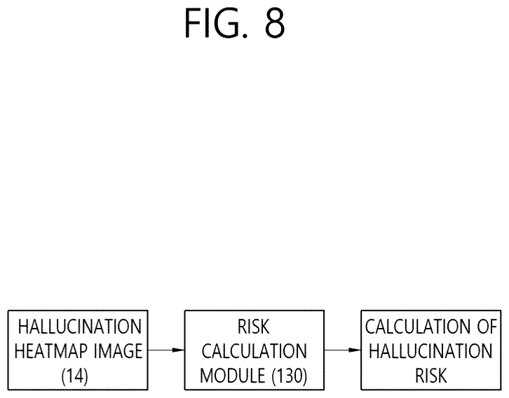

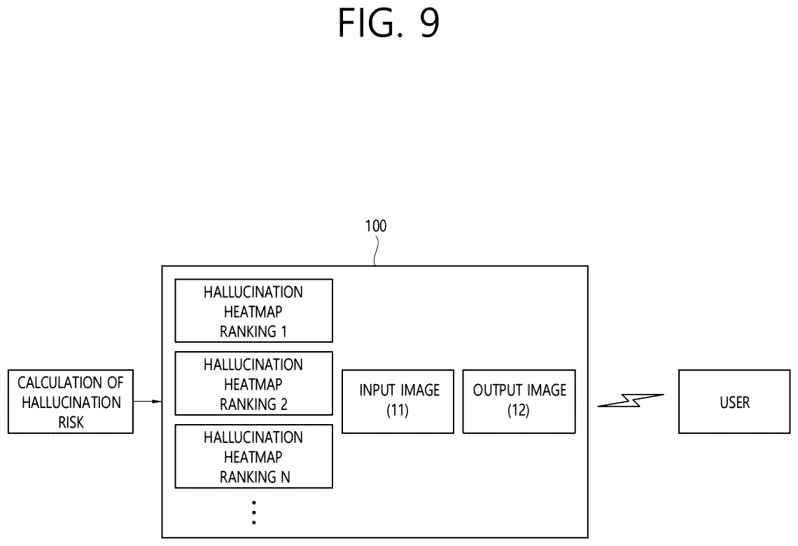

FIG. 8 is a conceptual view showing a method of calculating an enhancement risk based on a heatmap image in an auditing method using an image enhancement auditing apparatus according to an embodiment, and FIG. 9 is a flowchart showing a method of providing auditing information in an image enhancement auditing apparatus according to an embodiment.

As shown in FIGS. 8 and 9, the image enhancement auditing apparatus 100 according to an embodiment includes the risk calculation module 130. The risk calculation module 130 may calculate a hallucination risk of the heatmap image 14.

As described above, the heatmap refers to graphics in which values of data are converted into colors and visualized in the form of heat distribution. Thus, in the heatmap image 14 output from the deep learning model 121, the hallucination region in the comparison image 13 may be expressed in the form of heat distribution. For example, a region with a high probability of hallucination may be expressed in red, and a region with a moderate probability of hallucination may be expressed in green. In addition, a region with a low probability of hallucination may be expressed in blue.

Thus, the risk calculation module 130 may calculate the hallucination risk by using the maximum pixel value or the pixel value sum based on the colors in the heatmap image 14 provided from the heatmap generation module 120.

Then, the image enhancement auditing apparatus 100 sorts the plurality of heatmap images, in which the hallucination risks have been calculated, so as to provide the auditing information to a user. In this case, the image enhancement auditing apparatus 100 may sort the heatmap images, which have been provided by the risk calculation module 130, in order of high hallucination risk.

Further, the image enhancement auditing apparatus 100 provides the input image 11, the output image 12, and the heatmap image 14 to a user by a preset communication method. Thus, a user compares the input image 11, the output image 12, and the heatmap image 14, thereby auditing whether a hallucination is generated in the image enhancement process of the medical image enhancement device 10.

Thus, the apparatus and method for the auditing of the artificial intelligence-based medical image enhancement makes it possible to identify whether the hallucination is involved in the results output from the medical image enhancement device, thereby ensuring the safe operations of the medical image enhancement device.

Although a few embodiments of the disclosure have been described above and illustrated in the accompanying drawings, the embodiments should not be construed as limiting the technical spirit of the disclosure. The scope of the disclosure is limited only by the subject matters disclosed in the appended claims, and the technical spirit of the disclosure may be modified and changed in various forms by a person having ordinary knowledge in the art. Therefore, such modification and change obvious to those skilled in the art will fall within the scope of the disclosure.

What is claimed is:

1. A method of auditing of artificial intelligence-based medical image enhancement, the method comprising:
performing preprocessing to generate a comparison image based on an input image and an output image received from a medical image enhancement device;
generating a heatmap image to generate a hallucination heatmap image comprising a hallucination region in the comparison image by inputting the comparison image to a deep learning model trained in advance;
calculating an error risk to calculate a hallucination risk for the hallucination region based on pixel values of the hallucination heatmap image; and
providing auditing information to provide the auditing information for auditing accuracy of the output image based on the calculated hallucination risk to a user.

2. The method of claim 1, wherein the performance of the preprocessing comprises:
receiving the input image and the output image;
generating a difference image between the input image and the output image;
applying a convolution operation with a preset kernel to the difference image; and
generating the comparison image by applying a predefined threshold to a result of the convolution operation.

3. The method of claim 1, wherein the performance of the preprocessing comprises:
receiving the input image and the output image;
calculating a local structural similarity index measure (SSIM) for each pixel of the input image and the output image; and
generating the comparison image by generating an image based on the local SSIM and applying a predefined threshold to the local SSIM.

4. The method of claim 1, wherein the generation of the heatmap image comprises:
receiving the comparison image; and
applying the comparison image to the deep learning model, which divide the comparison image into a normal image and an abnormal image, to output a hallucination region in a heatmap format.

5. The method of claim 4, further comprising: in training the deep learning model,
generating a plurality of output images by inputting the plurality of input images to the medical image enhancement device,
labeling the plurality of output images divisionally with normal images and abnormal images to make up a training data set, and
inputting the training data set to the deep learning model so that the deep learning model can be trained to output a hallucination heatmap image for the hallucination region.

6. The method of claim 5, wherein, in the training, the deep learning model is trained to compare the normal image and the abnormal image, and identify a hallucination region based on a changed region in the abnormal image compared to the normal image.

7. The method of claim 6, wherein the deep learning model is provided as a single model to identify the hallucination region from the comparison image upon the comparison image being provided, and apply a heatmap format to the hallucination region.

8. The method of claim 7, wherein the deep learning model comprises:
a first deep learning model configured to identify the hallucination region from the comparison image upon the comparison image being provided; and
a second deep learning model linked to the first deep learning model and configured to apply a heatmap format to the hallucination region.

9. The method of claim 1, wherein the calculation of the error risk comprises calculating the hallucination risk based on at least one of a maximum pixel value or a pixel value sum of the hallucination heatmap image.

10. The method of claim 1, wherein the provision of the auditing information comprises:
sorting the plurality of hallucination heatmap images, in which the hallucination risks have been calculated, in order of high hallucination risk, and
providing the input image, the output image, and the hallucination heatmap image, in which the hallucination risk is high, to the user.

11. An apparatus for auditing of artificial intelligence-based medical image enhancement, the apparatus comprising:
a preprocessing module configured to generate a comparison image based on an input image and an output image received from a medical image enhancement device;
a heatmap generation module configured to generate a hallucination heatmap image comprising a hallucination region in the comparison image by inputting the comparison image to a deep learning model trained in advance; and
a risk calculation module configured to calculate a hallucination risk for the hallucination region based on pixel values of the hallucination heatmap image,
wherein auditing information for auditing accuracy of the output image based on the calculated hallucination risk is provided to a user.

12. The apparatus of claim 11, wherein the preprocessing module is configured to:
receive the input image and the output image;
generate a difference image between the input image and the output image;
apply a convolution operation with a preset kernel to the difference image; and
generate the comparison image by applying a predefined threshold to a result of the convolution operation.

13. The apparatus of claim 11, wherein the preprocessing module is configured to:
receive the input image and the output image;
calculate a local structural similarity index measure (SSIM) for each pixel of the input image and the output image; and
generate the comparison image by generating an image based on the local SSIM and applying a predefined threshold to the local SSIM.

14. The apparatus of claim 1, wherein the heatmap generation module is configured to:
receive the comparison image; and
apply the comparison image to the deep learning model, which divide the comparison image into a normal image and an abnormal image, to output a hallucination region in a heatmap format.

15. The apparatus of claim 14, wherein, in training the deep learning model,
a plurality of output images is generated by inputting the plurality of input images to the medical image enhancement device,
the plurality of output images are divisionally labeled with normal images and abnormal images to make up a training data set, and
the training data set is input to the deep learning model so that the deep learning model can be trained to output a hallucination heatmap image for the hallucination region

16. The apparatus of claim 15, wherein the deep learning model is trained to compare the normal image and the abnormal image, and identify a hallucination region based on a changed region in the abnormal image compared to the normal image.

17. The apparatus of claim 16, wherein the deep learning model is provided as a single model to identify the hallucination region from the comparison image upon the comparison image being provided, and apply a heatmap format to the hallucination region.

18. The apparatus of claim 17, wherein the deep learning model comprises:
a first deep learning model configured to identify the hallucination region from the comparison image upon the comparison image being provided; and
a second deep learning model linked to the first deep learning model and configured to apply a heatmap format to the hallucination region.

19. The apparatus of claim 11, wherein the risk calculation module is configured to calculate the hallucination risk based on at least one of a maximum pixel value or a pixel value sum of the hallucination heatmap image.

20. The apparatus of claim 11, wherein, in providing the auditing information,
the plurality of hallucination heatmap images, in which the hallucination risks have been calculated, are sorted in order of high hallucination risk, and
the input image, the output image, and the hallucination heatmap image, in which the hallucination risk is high, are provided to the user.
